(19) United States
(12) Patent Application Publication
(10) Pub. No.: US 2009/0017173 A1
(43) Pub. Date: Jan. 15, 2009

(54) AUTOMATIC PROCESS CONTROL OF CONVEYORIZED OVENS FOR IMPROVED BAKING EFFICIENCY AND PRODUCT UNIFORMITY

(76) Inventors: Austin J. Kozman, Dallas, TX (US); Robert W. Middleton, JR., Plano, TX (US); Keith A. Dietz, Allen, TX (US); Jay K. Smith, Lowry Crossing, TX (US)

Correspondence Address: MICHAEL A. O'NEIL, P.C., 5949 SHERRY LANE, SUITE 820, DALLAS, TX, 75225, US

(21) Appl. No.: 12/170,134

(22) Filed: Jul. 9, 2008

Related U.S. Application Data
(60) Provisional application No. 60/949,581, filed on Jul. 13, 2007.

(51) Int. Cl. G01N 33/02

(52) U.S. Cl. 426/233

(57) ABSTRACT

An automated process control system for conveyorized bakery ovens comprises measuring the moisture level within the oven; measuring the rate of flow of fuel into the oven; measuring the moisture level of bakery products exiting the oven; measuring the color and geometry of bakery products exiting the oven; and regulating the volumetric flow of exhaust gases through the exhaust ductwork of the oven in accordance with the foregoing measurements.

### CROSS-REFERENCE TO RELATED APPLICATION

Applicant claims priority based on U.S. Provisional Patent Application Ser. No. 60/949,581 filed Jul. 13, 2007, the entire content of which is incorporated herein by reference.

### DESCRIPTION OF THE DRAWINGS

FIG. 1 is a flow chart illustrating the invention.

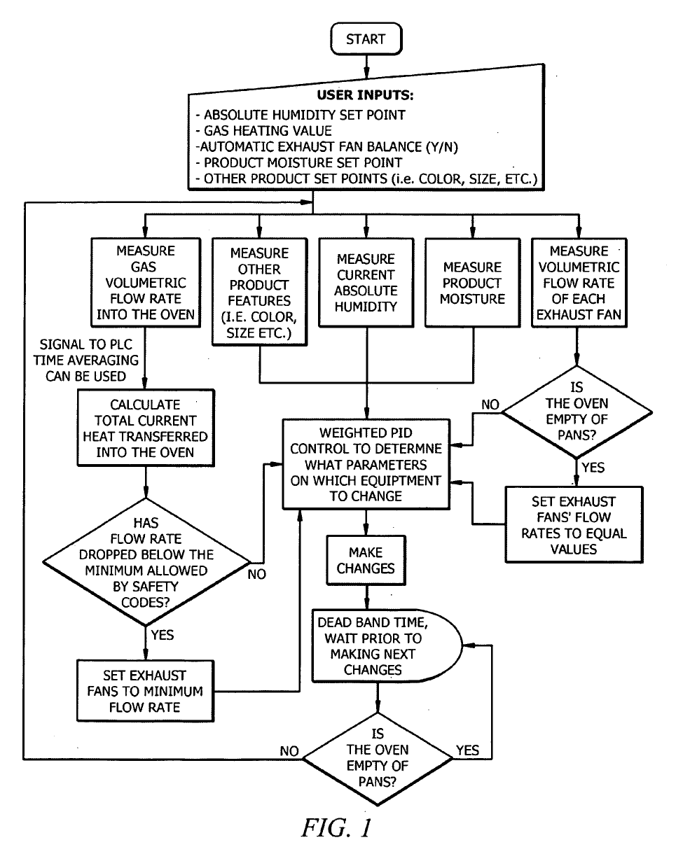

### DETAILED DESCRIPTION

As energy costs continue to rise bakeries are increasingly concerned about the energy efficiency of their equipment, in particular ovens. As is well known, ovens are generally the largest single consumer of energy in the bakery (whether they are gas-fired, electric, microwave, thermal fluid, etc).

Experimental and heat transfer analysis of the heat loss through the exhaust stacks of ovens (i.e. the need to heat up replacement air drawn into the ovens from the bakery ambient) can be as high as 45-50% of the total heat required to bake a pound of product (average of approximately 39.5%). This is a significant source of heat loss in the bakery, and some bakeries have incorporated heat recovery systems (which work best if implemented at the exit of the oxidizer) to recover some of the otherwise lost heat for other plant uses, e.g, proofers, boilers, hot water heaters, etc. However, the present invention addresses automatically reducing oven heat loss rather than recouping the energy down stream via automatic process control (APC), which can also be used to automatically tune the oven for product that is out of specification as to color, shape, moisture, etc.

Reducing the exhaust fan frequency (and consequently the volumetric flow rate of the exhaust leaving the oven) can reduce exhaust stack heat loss and energy consumption (similar effects can be obtained by flow dampeners in the exhaust ducts or other mechanical flow restrictions with automatic adjustment such, as motorized or pneumatic dampeners). The amount of reduction depends on the fan curve and the current operating point. However, care must be used because reduced exhaust levels can impact the moisture level of the product and other product characteristics. Also, if the exhaust flow is reduced too much the oven atmosphere can become saturated and moisture from the air will condense onto the crown of the product resulting in undesirable white blotches and spots. Finally, the exhaust flow level should never be reduced below the value required to safely exhaust the products of combustion (183 SCFM per 1.0 MBTU/hr rating of the oven per NFPA 86 Section 9.2.6.3).

The system and control algorithm disclosed herein automatically keep the oven exhaust level at the absolute minimum level required for baking a quality product, safety regulation compliance, flame stability, etc. or at an arbitrary level defined by the customer.

### High Temperature Humidity Sensor/Transmitter

Humidity sensors designed to operate in high temperature environments (up to 2400° F.) are installed directly through the oven wall to measure the absolute humidity or moisture level of the oven. The sensor can employ the indirect oxygen displacement principle, direct measurement of the partial pressure of water vapor in the oven compared to the total pressure, etc. A transmitter sends the absolute humidity signal from the sensor to a programmable logic controller (PLC). The PLC monitors the oven moisture and compares it to the desired setpoint for a selected product (as defined by the user); and adjusts the volumetric flow of the exhaust fans as required (either by automatic damper adjustments or by changing the operating speed/frequency of the fan). The sensor is placed in an area away from the conveyor openings, direct burner flame impingements, and other areas in the oven that may have a different local moisture level as compared with the bulk of the air in the oven. Multiple sensors can be used and averaged to obtain a better approximation of the total moisture content throughout the oven, especially in large ovens.

### Exhaust Flow Sensor/Transmitter

One or more sensors are mounted in the ductwork of each exhaust fan to measure the average and/or maximum volumetric flow rate of the fan at any selected time. The transmitter sends the flow rate to the PLC which monitors the combined flow rates from all active exhaust fans to assure that the continued/low rate has not fallen below the minimum required safety value. If the combined flow rate is too low the PLC causes the fans to operate at the speed required to obtain the minimum flow rate (depending upon ambient conditions). The transmitters also have at least one relay which provides a proof of flow interlock as required by most safety codes. Another safety benefit resulting from use of the sensor is achieved by coupling the measured instantaneous flow rates to a computerized totalizer to ensure the proper volume has been purged from the oven prior to an ignition attempt. Also, the volumetric flow rate for each fan can be displayed to an operator to assure that the fans are balanced and thereby optimizing the oven air flow pattern. The PLC can also automatically balance the fans. The sensor can be thermal mass, differential pressure, turbine, plunger, paddle wheel, ultrasonic, etc. Using a photo eye, proximity sensor, etc. or other pan interval detection (PInDe) methods to detect pans entering and exiting the oven the PLC can determine that the oven is empty and begin to slow the exhaust fans to their lowest possible safe value to minimize energy consumption in down periods.

The PInDe is too slow to respond large prolonged pan gaps in the system. So, when a pan gap is detected that is longer than a preset time interval the oven goes into its automatic temperature control mode. It will go into over temperature mode by lowering the set point temperature (burners will go off and those that remain on will go to low fire because of the PInDe duty). Once pans re-enter the system automatically returns to its previous operating point. This prevents the first and last pans from being burnt in the oven.

### Gas Flow Sensor/Transmitter

A gas flow sensor is mounted in the main gas feed pipe to the oven downstream of the main gas regulator and upstream of the first main safety shut-off valve of the oven (the exact location depends on the type of sensor used). The sensor measures the average and/or maximum volumetric flow rate of gas entering the oven. The transmitter sends the flow rate to the PLC which multiplies the flow rate by the heating value of the incoming gas to calculate the current energy consumption level of the oven. The calculated energy level determines amount of air that must be exhausted from the oven which is the minimum valued used for the comparison described above in the Exhaust Flow Sensor/Transmitter section. Another benefit of the gas flow sensor is that the user can track instantaneous as well as average energy consumption over time.

### Near Infrared (NIR) Sensor/Transmitter

Using optics the NIR sensor detects surface level moisture in a wide variety of products. In the bakery industry the NIR sensor is used to perform in-situ measurements of crust moisture level. The crust has a different moisture level as compared to the inner crumb of the product, but the two are generally directly proportional (i.e. the drier the crust the direr the crumb and vice-versa). A product moisture transmitter measures product as it exits the oven and sends the moisture level to the PLC which adjusts the exhaust level to match the desired set point based on the control algorithm.

### Color Sensor/Vision System

Other sensors are used to collect product information which is compared to desired specifications in addition to moisture. For example, digital cameras are used to capture both the color and geometry (size, shape, etc.) of each product. Due to the relatively short process time in the oven the digital camera is placed as close as possible to the exit of the oven. For optimal performance the sensors should be located no further away than at the exit of the depanner. However, in most commercial bakeries the inspection systems are located at the end of the line adjacent to the packaging equipment so that by the time the inspection systems detect a trend shift in the product it is too late to make an automatic adjustment that will correct the remaining product. Therefore, existing inspection systems do not provide the closed loop Proportional Interval Derivative (PID) control system of the present embodiment described above for automatic process control.

Depending what sensor is used various adjustments can be made to correct the oven or even upstream equipment such as the proofer. For example, within the oven operation of the recirculation system can be adjusted to: modify the flow rate of the air (inverter driven fan), air distribution (dampers), modify the amount of steam (moisture), increase heat in various zones by providing each zone with a pressure transmitter that transmits the combustion air pressure to the PLC, modify temperature and time, modify exhaust fan speed, etc. Similarly within the proofer operational characteristics can be regulated by modifying time, temperature, relative humidity, etc. Operation of the depanner can also be maximized by modifying belt height, vacuum pressure, etc.

### Control Algorithm

PID control is customized by weighting the importance of various factors and an algorithm to ensure the proper process variable is changed for a given product defect. There is also a given dead band time between changes while the oven stabilizes prior to making another change to prevent the system from oscillating and one variable impacting another. In most instances the variables are changed in series to get to the final control point and minimize uncertainty in parallel adjustment. If, after a given period of time and after exhausting all the alternatives in the algorithm for a given defect or problem the desired result has not been obtained the PLC issues a report notifying the operator that the system has made all of the programmed adjustments and nothing has changed, so the problem must be in an upstream process that the system does not control (i.e. the mixer, fermentation room, etc.).

The above-described process control system may further include one or more of the following:

monitoring a O2, CO, or combustion analyzer and periodically adjusting the upstream needling valve to maintain the oven at the best possible stochiometric combustion;

monitoring horsepower/amperage on the conveyor drives to automatically start the oiler;

monitoring seized bearings and marking the chain for replacement at the next down time;

monitoring magnet gauss levels in the magnets to locate and signal defective conveyor magnet(s);

monitoring the amount of lubricant fired by each nozzle of the automatic lubrication system to ensure that each nozzle is not clogged and is depositing the correct amount of oil;

ultrasonic track monitoring to measure the depth of wear grooves in the track to signal the operator when replacement is needed prior to failure; and

monitoring tension in drive chains via a strain gauge, force sensor, or displacement sensor and notifying operator when an adjustment is required. Or, if pneumatic changing the electronic regulator in the take-up automatically.

### EXAMPLE

A bakery has established the following set points for a high speed bun line:

Temperature=450 F

Bake time=8 min.

Absolute Humidity=38% Weight=0.10

Crust moisture content=25% Weight=0.20

Color (L* or intensity)=120 Weight=0.70

Diameter=4.0″ Weight=0.00

Fans Matched=Yes

The gas flow rate is measured and the energy value calculated. Assume that the bun line is running at 50% capacity and that natural gas is flowing at 2000 CFH yielding 2 MBTU/hr. The minimum exhaust level for safety code compliance is 366 SCFM. The exhaust fan flow rates are 1192 SCFM and 1225 SCFM (nominal 1200 SCFM fans are used), i.e., the exhaust gases flow rate is well above the minimum exhaust level. If it was not the operating speeds of the fams must be increased until each fan is operating at or above 193 SCFM (a safety factor of 5% assumed, but can be changed). Because the fans need to be balanced, the operating speed of the slower fan is increased and the operating speed of the faster fan is decreased until the operating speeds of the two fans is within 1% of each other. When the two fans are operating at the same speeds (within 1%), the current value of the parameters as measured by the sensors is:

Absolute Humidity=20%

Crust moisture content=15%

Color (L* or intensity)=160

Diameter=4.05″

Since the diameter has a weight of zero it is ignored. Using the weights assigned to absolute humidity and crust moisture, the fan speeds are reduced to exhaust less air. The PID values also determine how quick and fast this adjustment is; assume a fan speed reduction of 1200 SCFM or 600 SCFM per fan. Color is the most critical factor and it supersedes everything else, but its change requires an increase in temperature and bake time to make the product darker. Again, this depends on the PID parameters, but assume an increased time of 0.3 min. and an increased temperature of 7° F. At this point there is a dead band time before the next reading can be taken (user has specified 5 min).

After 5 min. the new values are:

Absolute Humidity=30%

Crust moisture content=19%

Color (L* or intensity)=110

Diameter=4.08″

The trend is still the same as to moisture so similar changes are made, except now the fan speed change may drop below the minimum exhaust rate so even though PID calculates 150 CFM for each fan, the fans are set at 193 SCFM. The buns are now too dark, so the PID adjusts time and temperature back down . . . assume 0.1 min. and 3° F. Then another dead band occurs.

After 5 min. the new values are:

Absolute Humidity=40%

Crust moisture content=37%

Color (L* or intensity)=120

Diameter=4.08″

At this point the moisture level is too high so the opposite is true. The fan speeds is increased, for example to 450 SCFM each. The color is good so the duty on the PID would be close to 0% and no change would be made. At this point another dead band occurs. However, before the dead band time is completed a big pan gap occurs resulting in initiation of the over temp and min exhaust routine. When pans are delivered again the normal control resumes.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. An automatic process control system for a conveyorized bakery oven comprising the steps of:
a. measuring the moister level within the oven;
b. measuring the volumetric flow of fuel into the oven;
c. measuring the moisture level of bakery products exiting the oven;
d. measuring color and geometry of bakery products exiting the oven;
e. measuring the volumetric flow of exhaust gases flowing through the exhaust ductwork comprising the oven;
f. utilizing the measurements comprising steps a., b., c., and d. to determine the optimum volumetric flow of the exhaust gases through the exhaust ductwork comprising the oven; and
g. regulating the volumetric flow of gases through the exhaust ductwork comprising the oven in accordance with the determination of step f.
